(19) United States
(12) Patent Application Publication
(10) Pub. No.: US 2013/0331786 A1
(43) Pub. Date: Dec. 12, 2013

(54) Device for Controlling a Penetration Depth of Injection Needle

(71) Applicant: Verena Hofmann, Frankfurt am Main (DE)

(72) Inventor: Verena Hofmann, Frankfurt am Main (DE)

(73) Assignee: Sanofi-Aventis Deutschland GmbH, Frankfurt am Main (DE)

(21) Appl. No.: 13/880,532

(22) PCT Filed: Oct. 25, 2011
(86) PCT No.: PCT/EP11/68591

§ 371 Date: Aug. 22, 2013

(30) Foreign Application Priority Data
Oct. 25, 2010 (EP) 10188655.4

(51) Int. Cl. A61M 5/46

(52) U.S. Cl. CPC A61M 5/46
USPC 604/117

(57) ABSTRACT

According to the invention, a device for controlling a penetration depth to which an injection needle penetrates a body of a human or an animal comprises
a cylindrical first sleeve,
an injection needle that protrudes a distal end of the first sleeve in a distal direction and
a cylindrical second sleeve that receives the first sleeve.

The penetration depth of the injection needle corresponds to an axial translation of the first sleeve with respect to the second sleeve. The axial translation is adjustable by a screw coupling.

### CROSS REFERENCE TO RELATED APPLICATIONS

The present application is a U.S. National Phase Application pursuant to 35 U.S.C. §371 of International Application No. PCT/EP2011/068591 filed Oct. 25, 2011, which claims priority to European Patent Application No. 10188655.4 filed Oct. 25, 2010. The entire disclosure contents of these applications are herewith incorporated by reference into the present application.

### FIELD OF INVENTION

### Background

It is well known that various medications, drugs or, in particular, vaccines need to be disposed into the skin of a patient or beneath the skin of a patient at a certain depth. Injection needles suitable for intramuscular, subcutaneous, intradermal or transcutaneous injections thus comprise different lengths. These injection needles may be directly connected to an injection device or may be connected to a needle hub that is releasably attached to the injection device by, for example, a luer lock connection.

### SUMMARY

It is an object of the present invention to provide a device for controlling a penetration depth that allows for a quick and easy adjustment of the desired penetration depth.

Furthermore, it is an object of present invention to provide a device for controlling a penetration depth that allows for a choice of the penetration depth amongst a set of pre-defined values.

The object is achieved by a device for controlling a penetration depth according to claim 1.

Preferred embodiments of the invention are given in the dependent claims.

In the context of this specification, the terms distal and proximal are defined from the point of view of a person performing an injection. Consequently, a distal direction refers to a direction pointing towards the body of a patient receiving an injection and a distal end defines an end of an element that is directed towards the body of the patient. Respectively, the proximal end of an element or the proximal direction is directed away from the body of the patient receiving the injection and opposite to the distal end or distal direction.

According to the invention, a device for controlling a penetration depth to which an injection needle penetrates a body of a human or an animal comprises
a first sleeve,
an injection needle that protrudes a distal end of the first sleeve in a distal direction and
a second sleeve that receives the first sleeve. The penetration depth of the injection needle corresponds to an axial translation of the first sleeve with respect to the second sleeve. The axial translation is adjustable by a screw coupling.

The device provides a simple means to adjust the penetration depth of an injection needle and comprises only a few parts preferably made from a plastics material. The device can thus be economically produced in high quantities and is adapted to be used with disposable injection devices, like, for example, one-way syringes.

Said first sleeve and/or said second sleeve can be circular in a further embodiment. This allows for compact design.

Furthermore, the device can be used to adjust the penetration depth of the injection needle within a wide range. In particular, the penetration depth can be adapted to meet the demands for an intradermal, a transcutaneous, a subcutaneous or an intramuscular injection. The device is suited for various medical applications and, in particular, may be used in combination with an injection device delivering a vaccine into or beneath the skin of a person.

The thread connection comprises
an outer thread formed into an outer surface of the first sleeve and
an adjustment collar rotatably arranged with respect to the second sleeve. The adjustment collar comprises an inner thread formed into an inner surface of the adjustment collar that corresponds to the outer thread. The inner thread of the adjustment collar engages the outer thread of the first sleeve. The axial translation of the first sleeve with respect to the second sleeve is adjusted by actuating the adjustment collar.

The injection needle may be retracted and extended by manually turning the adjustment collar, whereby the direction of the movement of the injection needle depends on the orientation of the rotation. The device provides a simple and reliable mechanism to adjust penetration depth to a desired depth.

According to a possible embodiment of the invention, the first sleeve comprises at least one first locking cut-out and the adjustment collar comprises a second locking cut-out. The first and the second locking cut-out receive a locking peg to retain the first sleeve with respect to the second sleeve in a pre-determined position. The first sleeve in the pre-determined position is axially translated with respect to the second sleeve, wherein the axial translation corresponds to the penetration depth of the injection needle. As the axial translation is fixed by the locking peg engaging the first and the second locking cut-out, an inadvertent alteration of the adjusted penetration depth is avoided throughout the injection. In particular, the injection needle may be safely inserted into the skin or beneath the skin of a patient to the desired penetration depth.

According to another possible embodiment of the invention, the adjustment collar comprises a plurality of second locking cut-outs that are angularly displaced from each other. The first sleeve may be retained with respect to the second sleeve by a single locking peg engaging one of the second locking cut-outs and the first locking cut-out. The plurality of angular displaced second locking cut-outs allow for a retention of the first and the second sleeve independent of the angular orientation of the first and the second sleeve relative to each other. However, the pitch of the screw coupling determines the axial translation of the first sleeve with respect to the second sleeve and thus the penetration depth of the injection needle. Therefore, the adjusted penetration depth depends on the angular orientation of the first and the second sleeve relative to each other. The plurality of second locking cut-outs thus provides a means to lock the device to the desired penetration depth.

According to yet another possible embodiment of the invention, the first sleeve comprises a set of first locking cut-outs. The set of first locking cut-outs comprises a plurality of first locking cut-outs. Each first locking cut-out of the set is axially displaced from another first locking cut-out of the set, so that the first sleeve may be retained with respect to the second sleeve in a plurality of pre-determined positions. The device can thus be used in various medical applications demanding different penetration depths that correspond to the different pre-determined positions. The penetration depth may be fixed to the desired depth as the device may be retained in each of the pre-determined positions.

According to yet another possible embodiment of the invention, the first sleeve comprises a plurality of sets of first locking cut-outs that are angularly displaced from each other along the lateral side of the cylindrical first sleeve. This ensures that the device may be adjusted and retained in one of the pre-determined positions independently of the angular orientation of the first sleeve relative to the second sleeve.

The locking peg may comprise a locking projection that latches to a locking notch of the first sleeve to irreversibly lock the first sleeve with respect to the second sleeve in the pre-determined position. The locking peg engaging the locking notch provides an additional safety feature that prevents an accidental release of the locking peg fixing the penetration depth.

According to a possible embodiment of the invention, the axial translation in the pre-determined position corresponds to a penetration depth of approximately 0.12 mm for an intradermal injection, 2 mm for a transcutaneous injection, 2.2 mm for a subcutaneous injection or 8.7 mm for a intramuscular injection. The device may be used in combination with various injection devices and has a broad range of applicability. In particular, the device may be used to set the penetration depth for an intradermal vaccination or for a subcutaneous injection of insulin or an insulin derivative.

According to another possible embodiment, a radial protruding indicator disc is formed to the injection needle. A transparent indicator means comprises at least one calibration mark. The displacement of the indicator disc with respect to the calibration mark indicates the penetration depth of the injection needle. The indicator disc and the transparent indicator means allow for a quick pre-adjustment of the desired penetration depth before locking the device into one of the pre-determined positions.

The indicator means may comprise a cone-shaped needle cover attached to or integrated to a distal end of the second sleeve. The needle cover has a central aperture, so that the injection needle is allowed to protrude through the central aperture in a distal direction. The cone-shaped needle cover is a simple means to pre-set a maximal penetration depth, as the maximal distance by which the injection needle protrudes the needle cover in the distal direction is limited by the radial protruding indicator disc abutting an inner surface of the cone-shaped needle cover.

According to another possible embodiment of the invention, a transparent needle cap covers the needle cover and the injection needle protruding through the central aperture by the axial length corresponding to the penetration depth prior the injection. The user of the device can visually verify the location of the indicator disc with respect to the calibration mark while the indicator means and the injection needle is covered by the needle cap. The device thus allows for a safe adjustment of the penetration depth prior an injection, whereby accidental needle stick injuries are avoided.

According to a possible embodiment, the injection needle comprises a proximal end protruding into an interior of the first sleeve from a distal end of the first sleeve. A cartridge containing a medication or drug is at least partially inserted into the first sleeve. The cartridge is sealed by a septum prior to use that is pierced by the proximal end of the injection needle. The device is particularly adapted to be used in combination with reusable injection devices such as a cartridge syringe.

According to another possible embodiment of the invention, the injection needle is integrated to a distal end wall of the first sleeve. As the device may be cost-efficiently produced in high quantities, the device may be disposed after a single first injection has been performed.

In an alternative embodiment, injection needle is affixed to a needle hub that is releasably attached to the distal end of the first sleeve. The device is adapted to be re-used. After an injection has been performed the needle hub holding the injection needle is detached from the distal end of the first sleeve. Preferably, the needle hub is attachable to the distal end of the first sleeve by a thread connection, a bayonet coupling, a snap-lock connection or, more particularly, a Luer-type connection.

Details of the present invention are described hereinafter. However, it should be understood that the detailed description and the specific examples indicate possible embodiments of the invention and are given by way of illustration only. Various changes and modifications of the illustrated embodiments within the spirit and scope of the invention are appreciated by those skilled in the art.

### BRIEF DESCRIPTION OF THE DRAWINGS

The present invention will be better understood from the detailed description given in the following. The accompanying drawings are given for illustrative purposes only and do not limit the scope of the present invention.

FIG. 1 shows a sectional view of an injection device comprising a device for controlling a penetration depth.

FIGS. 2, 3 show sectional views of a locking peg with locking projections.

### DETAILED DESCRIPTION

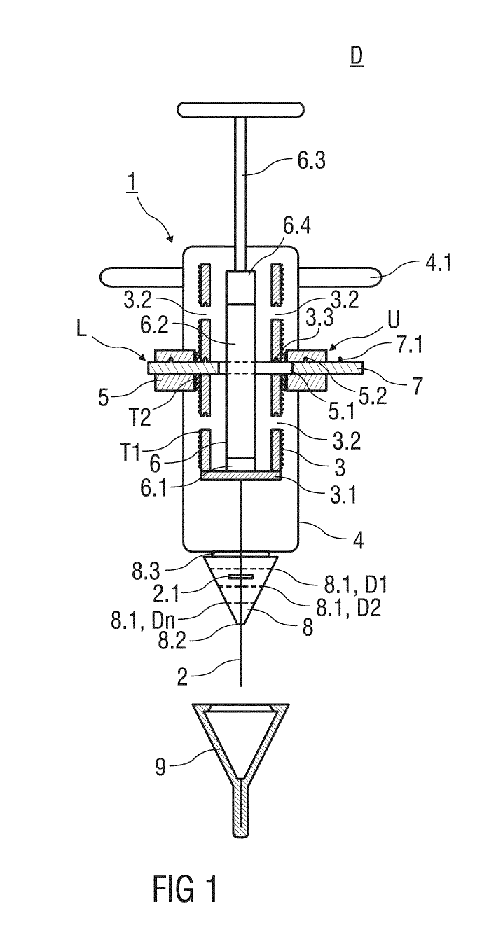

FIG. 1 schematically shows a sectional view of an injection device D comprising a device 1 for controlling a penetration depth D1 to Dn to which an injection needle 2 penetrates a human or animal body during an injection of a medication or drug. The device comprises a cylindrical first sleeve 3 retained within a cylindrical second sleeve 4. A circumferential adjustment collar 5 is rotatably arranged with respect to the second sleeve 4. An outer thread T1 is formed into an outer surface of the first sleeve 3 that engages a corresponding inner thread T2 formed into the inner surface of the adjustment collar 5.

FIG. 1 illustrates the operation principle of the injection device D schematically and is not to scale. In particular, the first sleeve 3 and the adjustment collar 5 of the device 1 are represented as enlarged views for better and more detailed illustration.

The second sleeve 4 may comprise gripping means 4.1 in the form of two radial protruding wings that support a user to perform an injection.

A rotation of the adjustment collar 5 with respect to the first sleeve 3 causes an axial translation of the first sleeve 3 relative to the second sleeve 4. The direction of the axial translation depends on the orientation of the rotation. The hollow injection needle 2 protrudes a substantially closed distal end of the first sleeve 3 in a distal direction. The injection needle 2 is fixed to the first sleeve 3, so that the injection needle 2 jointly moves with the first sleeve 3 when the adjustment collar 5 is actuated.

According to an embodiment of the invention, the injection needle 2 is integrated into a distal end wall 3.1 of the first sleeve 3. Alternatively, the injection needle 2 is affixed to a needle hub that is releasably attached to the distal end of the first sleeve 3. The releasable attachment of the needle hub to the distal end of the distal end of the first sleeve 3 is achieved by a screw connection, a bayonet coupling or a snap-lock connection. In particular, the needle hub may be connected to the distal end of the first sleeve 3 by a Luer-type connection.

In one embodiment of the invention, the injection needle 2 is double-ended and comprises a proximal end that protrudes into an interior of the first sleeve 3 from the distal end of the first sleeve 3. The proximal end of the injection needle 2 is adapted to pierce a septum 6.1 of a cartridge 6. The cartridge 6 is at least partially inserted into the first sleeve 3 and comprises an inner cavity 6.2 that is sealed by the septum 6.1. The inner cavity 6.2 contains a medication or drug that is expelled through the injection needle 2 by actuating a piston rod 6.3 connected to a piston 6.4. The piston 6.4 provides a fluid tight seal for a proximal end of the inner cavity 6.2 and moves within the inner cavity 6.2 when the piston rod 6.3 is pushed in the distal direction.

Alternatively, a pre-filled syringe is at least partially received within the first sleeve 3 in a manner that the injection needle 2 affixed to a distal end of the pre-filled syringe protrudes the first sleeve 3 in the distal direction. The medication or drug is contained within the pre-filled syringe and is expelled through the injection needle 4 during the injection.

A plurality of first locking cut-outs 3.2 is formed into the first sleeve 3 at pre-determined positions. A set of first locking cut-outs 3.2 comprises a plurality of first locking cut-outs 3.2, wherein each first locking cut-out 3.2 is axially displaced from another by pre-determined axial lengths. The set of first locking cut-outs 3.2 is aligned parallel to the axis of the cylindrical first sleeve 3.

FIG. 1 shows two sets of first locking cut-outs 3.2 formed into opposite sides of the cylindrical first sleeve 3. The sets of first locking cut-outs 3.2 are angularly displaced from each other by an angle of 180 degrees around the cylindrical axis of the first sleeve 3.

Alternatively, the first sleeve 3 comprises a plurality of sets of first locking cut-outs 3.2 that are angularly displaced from another by an angle of, for example, 30 degrees, 45 degrees, 90 degrees or 120 degrees.

The adjustment collar 5 comprises at least one second locking cut-out 5.1 with a diameter corresponding to a diameter of the first locking cut-out 3.2. A locking peg 7 is inserted in the second locking cut-outs 5.1. In an unlocked position U, the locking peg 7 protrudes in a radial outward direction from the adjustment collar 5. The locking peg 7 protrudes into the first locking cut-out 3.2 in a locked position L, so that the axial translation of the first sleeve 3 relative to the second sleeve 4 is fixed.

The first sleeve 3 and the second sleeve 4 can be locked in a plurality of pre-determined positions in which the first and second sleeve 3, 4 are axially translated relative to each other. The number of pre-determined positions is given by the number of axially displaced first locking cut-outs 3.2 of the first sleeve 3. The axial translation of the first sleeve 3 with respect to the second sleeve 4 in one of the pre-determined positions corresponds to a penetration depth D1 to Dn of the injection needle 2.

According to a possible embodiment of the invention, at least one of the pre-determined positions corresponds to a penetration depth D1 to Dn of approximately 0.12 mm for an intradermal injection, 2 mm for a transcutaneous injection, 2.2 mm for a subcutaneous injection or 8.7 mm for an intramuscular injection.

As shown in FIG. 1, two second locking cut-outs 5.1 are formed into opposite sides of the adjustment collar 5. The second locking cut-outs 5.1 are angularly displaced from each other by an angle of 180 degrees around the cylindrical axis. Each of the second locking cut-outs 5.1 contains one locking peg 7, whereas the first sleeve 3 may be locked with respect to the second sleeve 4 by pushing at least one of the locking pegs 7 radially inwards.

Alternatively, the adjustment collar 5 comprises a plurality of second locking cut-outs 5.1 angularly displaced from another. The number of second locking cut-outs 5.1 may correspond to the number of sets of first locking cut-outs 3.2. Furthermore, the angular displacement of the second locking cut-outs 5.1 may correspond to the angular displacement of the sets of first locking cut-outs 3.2.

The locking peg 7 comprises at least one locking projection 7.1 that latches to a corresponding locking notch 3.3 of the first sleeve 1 when the locking peg 7 is pushed radially inwards from the unlocked position U to the locked position L. The locking projection 7.1 latching to the locking notch 3.3 permanently locks the locking peg 7 in the locking position L.

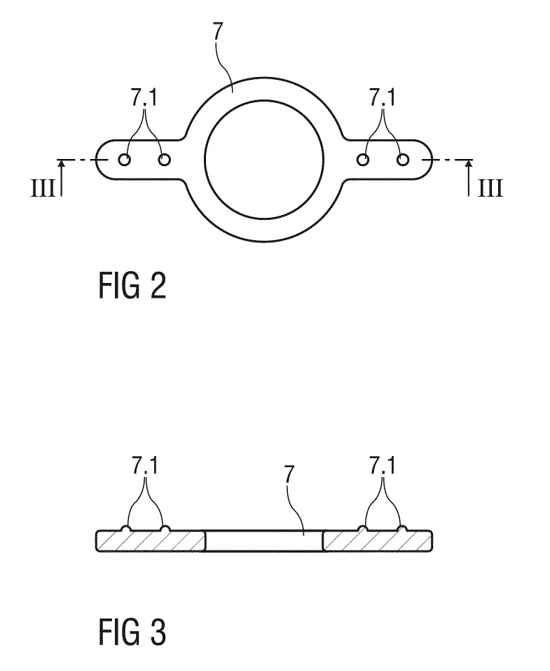

FIGS. 2 and 3 show sectional views of a possible embodiment of a locking peg 7 with locking projections 7.1.

Alternatively or additionally, the locking projection 7.1 may engage a retaining notch 5.2 to retain the locking peg in the unlocked position U. The locking projection 7.1 may be resiliently deflectable, so that the locking peg 7.1 is allowed to move radially inwards from the unlocked position U to the locked position L.

An indicator disc 2.1 is formed to the injection needle 2. The indicator disc 2.1 is made from a plastics material and is spaced away from a distal end of the injection needle 2 by a length that corresponds to or exceeds the maximal penetration depth Dn. The indicator disc 2.1 protrudes the elongate injection needle 2 in the radial outward direction and may be coloured for better recognition.

The device 1 for controlling a penetration depth Dl to Dn comprises a transparent and cone-shaped needle cover 8 as an indicator means. The needle cover 8 is attached to a distal end of the second sleeve 4 and comprises at least one circumferential calibration mark 8.1 and a central aperture 8.2. The displacement of the indicator disc 2.1 with respect to the calibration mark 8.1 indicates the penetration depth D1 to Dn of the injection needle 2.

As already mentioned herein above, FIG. 1 is not to scale. In particular, it is understood by a person skilled in the art that the axial displacement of the calibration marks 8.2 from each other must correspond to the axial displacement of the first locking cut-outs 3.2 from each other and to the different penetration depths D1 to Dn.

Additionally or alternatively, the indicator disc 2.1 may be sized to correspond to a diameter of the cone-shaped needle cover 8, so that the indicator disc 2.1 abuts an inner surface of the needle cover 8 when the injection needle 2 protrudes through the central aperture 8.2 by a length that corresponds to a maximal penetration depth Dn.

The needle cover 8 may comprise a plurality of calibration marks 8.1 that correspond to a penetration depth D1 to Dn of approximately 0.12 mm, 2 mm, 2.2 mm, and/or 8.7 mm.

A transparent needle cap 9 covers the injection needle 2 prior to use of the injection device D. The needle cap 9 is sized to cover the injection needle 2 protruding the central aperture 8.2 by a length that corresponds to the maximal penetration depth Dn. The needle cap 9 may partially cover the indicator means.

The needle cap 9 may be connected to the needle cover 8 by a snap-lock connection. The needle cap 9 may latch to a circumferential notch 8.3 to affix the needle cap 9 to the needle cover 8.

Alternatively, the needle cap 9 may be frictionally affixed to the needle cover 8.

The penetration depth D1 to Dn is adjusted by rotating the adjustment collar 5, whereby the first sleeve 3 is axially translated with respect to the second sleeve 4. The injection needle 2 jointly moves with the first sleeve 3, whereby the transparent needle cap 9 covers the cone-shaped needle cover 8 and the injection needle 2. When the indicator disc 2.1 is aligned with the calibration mark 8.1, one of the locking pegs 7 is pushed radially inwards to engage one of the first locking cut-outs 3.2 in one of the pre-determined positions that corresponds to the desired penetration depth D1 to Dn. The needle cap 9 is removed and the injection needle 2 is inserted into or beneath the skin of the patient. The medication or drug is expelled by pushing the piston rod 6.3 in the distal direction.

1. A device for controlling a penetration depth to which an injection needle penetrates a body of a human or an animal, wherein the device comprises
a first sleeve,
an injection needle that protrudes a distal end of the first sleeve (3) in a distal direction and
a second sleeve that receives the first sleeve,
wherein penetration depth of the injection needle corresponds to an axial translation of the first sleeve with respect to the second sleeve, whereby the axial translation is adjustable by a screw coupling characterized in that a radial protruding indicator disc is formed to the injection needle and a transparent indicator means with at least one calibration mark indicates the penetration depth of the injection needle.

2. A device according to claim 1, characterized in that the screw coupling comprises
an outer thread formed into an outer surface of the first sleeve,
an adjustment collar rotatably arranged with respect to the second sleeve,
wherein the adjustment collar comprises an inner thread formed into the inner surface of the adjustment collar that corresponds to the outer thread and the axial translation of the first sleeve with respect to the second sleeve is adjusted by actuating the adjustment collar.

3. A device according to claim 2, characterized in that the first sleeve comprises at least one first locking cut-out and the adjustment collar comprises a second locking cut-out, wherein the first and the second locking cut-out receive a locking peg to retain the first sleeve with respect to the second sleeve in a pre-determined position, wherein the first sleeve in the pre-determined position is axially translated with respect to the second sleeve.

4. A device according to claim 2, characterized in that the adjustment collar comprises a plurality of second locking cut-outs that are angularly displaced from each other.

5. A device according to claim 2, characterized in that the first sleeve comprises a set of first locking cut-outs that comprises a plurality of first locking cut-outs (3.2), wherein each first locking cut-out of the set is axially displaced from another first locking cut-out of the set, so that the first sleeve is retainable with respect to the second sleeve in a plurality of pre-determined positions.

6. A device according to claim 2, characterized in that the first sleeve comprises a plurality of sets of first locking cut-outs that are angularly displaced from each other.

7. A device according to claim 2, characterized in that the locking peg comprises a locking projection that latches to a locking notch of the first sleeve to lock the first sleeve with respect to the second sleeve in the pre-determined position.

8. A device according to claim 2, characterized in that the axial translation in the pre-determined position corresponds to a penetration depth of approximately 0.12 mm, 2 mm, 2.2 mm or 8.7 mm.

9. A device according to claim 1, characterized in that the indicator means comprises a cone-shaped needle cover attached to or integrated to a distal end of the second sleeve, wherein the needle cover has a central aperture, so that the injection needle is allowed to protrude through the central aperture in a distal direction.

10. A device according to claim 9, characterized in that a transparent needle cap covers the needle cover and the injection needle protruding through the central aperture by a length corresponding to the penetration depth.

11. A device according to claim 1, characterized in that the injection needle comprises a proximal end protruding into an interior of the first sleeve from the distal end of the first sleeve, wherein the proximal end of the injection needle is adapted to pierce a septumof a cartridge.

12. A device according to claim 1, characterized in that the injection needle is integrated to a distal end wall of the first sleeve.

13. A device according to claim 1, characterized in that the injection needle is affixed to a needle hub that is releasably attached to the distal end of the first sleeve.
